(19) United States
(12) Patent Application Publication
(10) Pub. No.: US 2012/0183980 A1
(43) Pub. Date: Jul. 19, 2012

(54) KIT FOR AMPLIFYING DETECTED SIGNAL IN IMMUNOSENSOR AND METHOD FOR DETECTING TARGET ANTIGEN USING THE SAME

(75) Inventors: Tae Young KIM, Seoul (KR); Sun Kil KANG, Seoul (KR); Da Yeon KANG, Seoul (KR); Seung Mok HAN, Seoul (KR); Kyu Ho SONG, Seoul (KR); Guei Sam LIM, Seoul (KR); Ji Su KIM, Seoul (KR)

(73) Assignee: LG ELECTRONICS INC., Seoul (KR)

(21) Appl. No.: 13/351,816

(22) Filed: Jan. 17, 2012

(30) Foreign Application Priority Data
Jan. 17, 2011 (KR) 10-2011-0004690
Jan. 17, 2011 (KR) 10-2011-0004694

(51) Int. Cl. G01N 33/53; G01N 33/566; B82Y 5/00

(52) U.S. Cl. 435/7.94; 436/501; 435/7.9; 435/7.92; 977/773; 977/904

(57) ABSTRACT

Disclosed is a kit for amplifying detected signal in immunosensor and a method for detecting target antigen using the same according to the present invention, whereby a target antigen can be effectively detected even by a small amount of target antibody to thereby reduce nonspecific detection signal and to detect an amplified signal.

Pursuant to 35 U.S.C. §119 (a), this application claims the benefit of earlier filing date and right of priority to Korean Patent Application Nos. 10-2011-0004690 and 10-2011-0004694, filed on Jan. 17, 2011 and Jan. 17, 2011, the contents of which is hereby incorporated by reference in their entirety.

### BACKGROUND OF THE DISCLOSURE

1. Field

The teachings in accordance with the exemplary embodiments of this present invention generally relate to a kit for amplifying detected signal in immunosensor and a method for detecting target antigen using the same.

2. Background

A conventional immunosensor was used in such a manner that a captured antibody is placed on and fixed to a surface like a plate, an antigen is reacted, and an assayable label is attached to the detected antigen. However, the conventional method suffers from the following disadvantages:

First, nonspecific signals increase when an overdose of captured antibody is absorbed on the surface in order to increase detection signals;

Second, nonspecific signals increase when an overdose of detected antibody is admixed to reaction in order to increase detection signals;

Third, there is a limit in amplifying the detection signal, because the captured antibody fixed onto the surface can be coupled to only one or two detected antibodies; and

Fourth, there is a problem of adversely affecting performance of detected label, because a distance between a detected antibody connected by the detected label and a captured antibody.

Thus, a necessity of further using a new kit arises for amplifying a detected signal in the immunosensor according to the prior art.

### SUMMARY

The present invention has been made to solve disadvantages of the prior art and therefore an object of certain embodiments of the present invention is to provide a kit for amplifying detected signal in immunosensor.

Another object of certain embodiments of the present invention is to provide a method for detecting target antigen using a kit for amplifying detected signal in immunosensor.

Technical subjects to be solved by the present invention are not restricted to the above-mentioned description, and any other technical problems not mentioned so far will be clearly appreciated from the following description by the skilled in the art. That is, the present invention will be understood more easily and other objects, characteristics, details and advantages thereof will become more apparent in the course of the following explanatory description, which is given, without intending to imply any limitation of the disclosure, with reference to the attached drawings.

An object of the invention is to solve at least one or more of the above problems and/or disadvantages in whole or in part and to provide at least advantages described hereinafter. In order to achieve at least the above objects, in whole or in part, and in accordance with the purposes of the invention, as embodied and broadly described, and in one general aspect of the present invention, there is provided a kit for amplifying detected signal in immunosensor, the kit comprising: an antigen binder wherein a distal end of a first spacer is connected to a first antibody, and streptavidin is connected to a portion of the first spacer or the first antibody; and a signal amplifier wherein both distal ends of a second spacer bind to biotin and nanoparticle, and the nanoparticle binds to one or more detectable labels.

An immunosorbent assay method may be used for detection of a target antigen, particularly in capture-ELISA (enzyme-linked immunosorbent assay, enzyme-linked immunospecific assay). The capture-ELISA generally includes: (i) coating a capture-antibody on a surface of a solid substrate; (ii) reacting the capture-antibody and specimen (e.g., specimen including antibody that becomes a target); (iii) binding a resultant of the step (ii) to a detectable label generating a signal, and reacting specifically reacting detection antibody to the target antibody; and (iv) measuring a signal generated from the detectable label.

The present invention may characteristically use an antigen binder and a signal amplifier for amplifying the signal measured in (iv) step by changing detection antibody bound to a detectable label in step (iii).

In some exemplary embodiments, but not necessarily, the antigen binder may be configured in such a manner that a distal end of the first spacer is connected to the first antibody, and the other distal end of the first spacer is connected to streptavidin.

The first antigen may be defined by a detection antibody in the capture-ELISA, which may be specifically bound by a target antigen and antigen-antibody reaction.

In some exemplary embodiments, but not necessarily, the first spacer or second spacer may be comprised of one or more selected from a group consisting of polyethylene glycol, polyvinyl alcohol, polyacrylic acid, polymethacrylic acid, polyacrylamide and polyvinyl pyrrolidone.

Generally, a horizontal length occupied by the capture antibody on a surface in the capture-ELISA is approximately 15nm, and a vertical length to the capture antibody contacting the surface is approximately 5nm, such that the length of the first spacer or second spacer may be 30 Å˜60 Å, particularly 40 Å˜60 Å, and more particularly 50 Å.

In some exemplary embodiments, but not necessarily, the first spacer may be connected to the first antibody, and a distal end of the first spacer and the first antibody may be connected to streptavidin. The streptavidin may be connected to a distal end of the first spacer or the first antibody using a separate spacer.

The streptavidin is specific protein specifically bindable to biotin, and is comprised of four identical molecules. Thus, each molecule of streptavidin can be bound to one to four molecules of biotin. That is, it means that antigen binder including the streptavidin can be bound to four molecules of a signal amplifier including the biotin, which can be a specific element in signal amplification according to the present disclosure. The signal amplification will be explained later.

Furthermore, in some exemplary embodiments, but not necessarily, a distal end of the first spacer may be connected to the first antibody, and the other distal end of the first spacer may be connected to a detectable label.

The detectable label means an atom or a molecule configured to specifically detect a molecule including a label among identical types of molecules having no label, where the detectable label may include colored bead, antigen binder, enzyme, chromophore material, fluorescent material, phosphor material, electrically detectable molecule, molecule or quantum dot providing changed fluorescent—polarization or changed light spread. However, the detectable label is not limited thereto.

Furthermore, the label may be radio isotopes such as P³² and S³⁵, chemiluminescent compound, labeled bound protein, spectroscopic markers such as heavy metal atoms and dyes, and magnetic marker dyes. The dyes may include quinoline dye, triarylmethane dye, phthalein, azo dye and cyanine dye, for example. However, the label is not limited thereto.

The fluorescent material may include fluorescein, phycoerythrin, rhodamine, lissamine and Cy3 and Cy5 (Pharmacia). However, the fluorescent material is not limited thereto.

More preferably, the detectable label may be comprised of one or more selected from a group consisting of enzyme, for example, alkaline phosphatase, beta-galactosidase, horse radish, peroxidase and cytochrome P₄₅₀. However, the detectable label is not limited thereto.

In some exemplary embodiments, but not necessarily, the signal amplifier is configured in such a manner that both distal ends of the second spacer respectively bind to biotin and nanoparticle, where the nanoparticle may preferably be bound by one or more detectable labels.

The term of “nanoparticle” is a “ particle having one or more dimensions of the order of 1000 nm or less”, and preferably, a particle having a diameter in the range of 10 nm to 1000 nm. The ingredients comprising the nanoparticle may include metals such as Ag, Au, copper, aluminum, nickel, palladium and platinum, semiconductor materials such as DdSe, DdS, InAs and InP, an inactive substances such as polymeric materials including polystyrene, latex, acrylate and polypeptide, and may be comprised of one or more selected therefrom. However, the ingredients are not limited thereto.

The nanoparticle may bind to one or more detectable labels, and the number of bindable detectable labels may be determined by the size of the nanoparticle. The explanation of the detectable label has been already explained above.

The second spacer bound at both distal ends thereof by biotin, and nanoparticle bound to one or more detectable labels may include compound consisting of polyethylene glycol, polyvinyl alcohol, polyacrylic acid, polymethacrylic acid, polyacrylamide and polyvinylpyrrolidone. In some exemplary embodiments, length of the second spacer may be preferably 40 Å˜60 Å. The second spacer functions as a medium connecting the biotin to the nanoparticle bound to one or more detectable labels.

The signal amplifier functions to amplify a signal measured in the capture-ELISA (iv) step. To this end, the streptavidin and the biotin are mutually bound or bindable. As explained above, the amplification of signal is such that, due to specific binding between the streptavidin and the biotin, two or more signal amplifiers bind to one antigen binder, and preferably to four signal amplifiers bind to one antigen binder.

That is, the streptavidin of the antigen binder may bind to two or more signal amplifiers, and preferably four signal amplifiers, and the nanoparticle included in the signal amplifier binds to a plurality of detectable labels, such that signal strength is much stronger over a signal generated by a detectable label of one molecule relative to one target antigen.

In another general aspect of the present invention, there is provided a method for detecting target antigen using a kit for amplifying detected signal in immunosensor, the method comprising: contacting a target antibody, a target antigen and antigen binder; and detecting a signal generated from a detectable label of the antigen binder and the antigen binder.

In some exemplary embodiments, the target antigen may be fixed on a plate, where the plate may be comprised of such materials as polystyrene, Ag, carbon and indium tin oxide. However, the materials are not limited thereto.

The (iii) step of the method for detecting target antigen using a kit for amplifying detected signal uses the conventional capture-ELISA method except for contacting an antigen binder and the signal amplifier instead of detection antibody, which has been already described above.

The method may further include cleaning the antigen binder that is not specifically bound by the contact and the signal amplifier, subsequent to contacting the antigen binder and the signal amplifier. That is, the method may further include washing the antigen binder that is not bound by the contact, subsequent to contacting the antigen binder, and the method may further include cleaning the signal amplifier that is not bound by the contact, subsequent to contacting the signal amplifier. Through the washing process, a non-specifically generated signal may be reduced to amplify the signal more sensitively.

Other detailed matters according to exemplary embodiments will be included in the Detailed Description and drawings.

The kit for amplifying detected signal in immunosensor and a method for detecting target antigen using the same according to the present invention have an advantageous effect in that a target antigen can be effectively detected even by a small amount of target antibody to thereby reduce nonspecific detection signal and to detect an amplified signal.

Particular and preferred aspects of the invention are set out in the accompanying independent and dependent claims. Features from the dependent claims may be combined with features of the independent claims and with features of other dependent claims as appropriate and not merely as explicitly set out in the claims.

Although there has been constant improvement, change and evolution of devices in this field, the present concepts are believed to represent substantial new and novel improvements, including departures from prior practices, resulting in the provision of more efficient, stable and reliable devices of this nature.

The above and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference figures quoted below refer to the attached drawings.

### BRIEF DESCRIPTION OF THE DRAWINGS

FIG. 1 is a mimetic diagram illustrating an antigen binder according to an exemplary embodiment of the present invention;

FIG. 2 is a mimetic diagram illustrating a signal amplifier according to an exemplary embodiment of the present invention;

FIG. 3 is a schematic view illustrating a method in which a kit is used to amplify a detection signal in an immunosensor and to detect a target antigen according to an exemplary embodiment of the present invention;

FIGS. 4 and 5 illustrate a sandwich ELISA result and an electrode experiment result for detection of a target antigen using a kit according to an exemplary embodiment of the present invention;

FIG. 6 is a mimetic diagram illustrating an antigen binder according to another exemplary embodiment of the present invention;

FIG. 7 is a schematic view illustrating a method in which a kit is used to amplify a detection signal in an immunosensor and to detect a target antigen according to another exemplary embodiment of the present invention; and

FIGS. 8 and 9 are a sandwich ELISA result and an electrode experiment result for detection of a target antigen using a kit according to another exemplary embodiment of the present invention.

### DETAILED DESCRIPTION

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes. The dimensions and the relative dimensions do not correspond to actual reductions to practice of the invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

To more clearly and concisely describe and point out the subject matter of the claimed invention, the following definitions are provided for specific terms, which are used in the following description and the appended claims.

As may be used herein, the terms “substantially” and “approximately” provide an industry-accepted tolerance for its corresponding term and/or relativity between items.

It will be understood that, although the terms first, second, etc. may be used herein to describe various elements, these elements should not be limited by these terms. These terms are only used to distinguish one element from another. For example, a first region/layer could be termed a second region/layer, and, similarly, a second region/layer could be termed a first region/layer without departing from the teachings of the disclosure.

Descriptions of well-known components and processing techniques are omitted so as to not unnecessarily obscure the embodiments of the disclosure.

Words such as “thereafter,” “then,” “next,” “therefore”, “thus”, etc. are not intended to limit the order of the processes; these words are simply used to guide the reader through the description of the methods.

Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The singular forms “a” “an” and “the” include plural referents unless the context clearly dictates otherwise. Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified. Unless otherwise indicated, all numbers expressing quantities of ingredients, properties such as molecular weight, reaction conditions, so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least each numerical parameter should at least be construed in light of the number of reported significant digits and by applying ordinary rounding techniques.

That is, as used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Thus, for example, reference to “a component” can include a combination of two or more components; reference to “fluid” can include mixtures of fluids, and the like.

“Target,” as used herein, generally refers to the component of a biological sample that may be detected when present in the biological sample. The target may be any substance for which there exists a naturally occurring specific binder (e.g., an antibody), or for which a specific binder may be prepared (e.g., a small molecule binder). In general, the binder portion of the probe may bind to target through one or more discrete chemical moieties of the target or a three-dimensional structural component of the target (e.g., 3D structures resulting from peptide folding). The target may include one or more of peptides, proteins (e.g., antibodies, affibodies, or aptamers), nucleic acids (e.g., polynucleotides, DNA, RNA, or aptamers); polysaccharides (e.g., lectins or sugars), lipids, enzymes, enzyme substrates, ligands, receptors, antigens, or haptens.

As used herein, the term “binder” refers to a biological molecule that may non-covalently bind to one or more targets in the biological sample. A binder may specifically bind to a target. Suitable binders may include one or more of natural or modified peptides, proteins (e.g., antibodies, affibodies, or aptamers), nucleic acids (e.g., polynucleotides, DNA, RNA, or aptamers); polysaccharides (e.g., lectins, sugars), lipids, enzymes, enzyme substrates or inhibitors, ligands, receptors, antigens, haptens, and the like. A suitable binder may be selected depending on the sample to be analyzed and the targets available for detection. For example, a target in the sample may include a ligand and the binder may include a receptor or a target may include a receptor and the probe may include a ligand. Similarly, a target may include an antigen and the binder may include an antibody or antibody fragment or vice versa. In some embodiments, a target may include a nucleic acid and the binder may include a complementary nucleic acid. In some embodiments, both the target and the binder may include proteins capable of binding to each other.

As used herein, the term “antibody” refers to an immunoglobulin that specifically binds to and is thereby defined as complementary with a particular spatial and polar organization of another molecule. The antibody may be monoclonal or polyclonal and may be prepared by techniques that are well known in the art such as immunization of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM. Functional antibody fragments may include portions of an antibody capable of retaining binding at similar affinity to full-length antibody (for example, Fab, Fv and F(ab′).sub.2, or Fab′). In addition, aggregates, polymers, and conjugates of immunoglobulins or their fragments may be used where appropriate so long as binding affinity for a particular molecule is substantially maintained.

As used herein, immunosensors are a subset of biosensors. An immunosensor is a particular type of biosensor in which an antibody serves as the biological probe for a target antigen. An immunosensor is also commonly known as protein biosensor and works in a similar way as a DNA biosensor, except that the interaction between the antibody and the antigen is being converted into an analytical signal for measurement and detection.

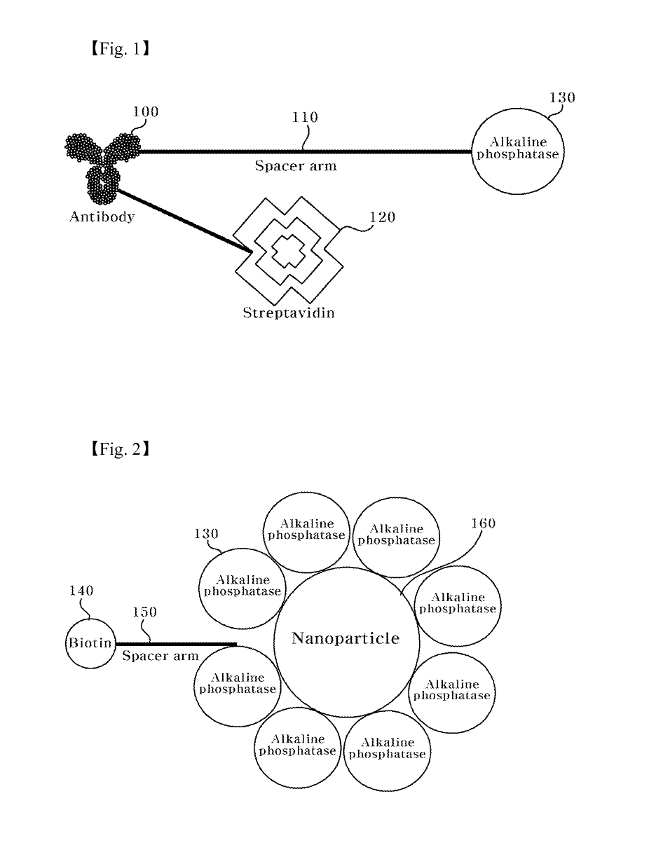

FIG. 1 is a mimetic diagram illustrating an antigen binder according to an exemplary embodiment of the present invention, FIG. 2 is a mimetic diagram illustrating a signal amplifier according to an exemplary embodiment of the present invention, and FIG. 3 is a schematic view illustrating a method in which a kit is used to amplify a detection signal in an immunosensor and to detect a target antigen according to an exemplary embodiment of the present disclosure.

Referring to FIGS. 1, 2 and 3, the kit for amplifying detected signal in immunosensor and a method for detecting target antigen using the same according to exemplary embodiments of the present disclosure will be explained and described with reference to the accompanying drawings.

FIG. 1 is a mimetic diagram illustrating an antigen binder according to an exemplary embodiment of the present invention, where a first antibody (100), a first spacer (110), streptavidin (120) and a detectable label (130) are illustrated in the diagram.

The first antibody (100) is an antibody binding to a target antigen, and connected to the detectable label (130) through the first spacer (110). Furthermore, the streptavidin (120) binds to a part of the first antibody (100) or the first spacer (120). The streptavidin (120) is characterized in that it is a substance comprised of four identical molecules, and is capable of binding to biotin of one to four molecules per molecule.

FIG. 2 is a mimetic diagram illustrating a signal amplifier according to an exemplary embodiment of the present invention, where a biotin (140), a second spacer (150), a nanoparticle (160) and a detectable label (130) are illustrated in the drawing.

The biotin (140) is a molecule configured to specifically bind to the streptavidin (120) of the antigen binder and binds to the nanoparticle (160) or the detectable label (130) through the second spacer (150). Furthermore, the nanoparticle (160) is bound by a plurality of detectable labels (130) to enable emission of much more amplified signal than that of the nanoparticle bound by a single detectable label.

FIG. 3 is a schematic view illustrating a method in which a kit is used to amplify a detection signal in an immunosensor and to detect a target antigen according to an exemplary embodiment of the present invention.

A target antibody (170) fixed to a surface (e.g., substrate) forms a complex by a target antigen (180) and antigen-antibody interaction, where if an antigen binder is brought into contact with the complex, an antigen-antibody interaction is generated by the first antibody (100) of the antigen binder and the target antigen (180).

The detectable label (130) bound to the first antibody (100) and the first spacer (110) can amplify a signal generated by the signal amplifier although a signal generated by the label is not great if the present disclosure is provided only with the antigen binder.

The antigen binder binds to the streptavidin (120), whereby a specific binding with the biotin (140) of the signal amplifier is possible. Due to the fact that the nanoparticle (160) connected to the biotin (140) through the seconds spacer (150) binds to a plurality of detectable labels (130), a signal having a high sensitivity can be detected by binding of one particle of antigen binder alone.

### First Exemplary Embodiment: Manufacturing Method of Antigen Binder and Signal Amplifier

A method for manufacturing an antigen binder is as follows:

First, one side of polyethylene glycol was bound by N-hydrosuccinimide estere group bindable with amine group, and the other side was bound by maleimide group bindable with sulfhydryl group, alkaline phosphatase was made to react with ethylmaleimide to restrict its polymer shape, alkaline phosphatase was covalent-bonded to hydrosuccinimide group of polyethylene glycol, and maleimide group of polyethylene glycol is covalently bonded to reduced hepatitis B antibody (manufactured by Arista). Then, sulfhydryl group connected to N-hydrosuccinimide group was additionally covalently bonded to the amine group of reduced hepatitis B antibody. The streptavidin was bounded to N-hydrosuccinimide group of polyethylene glycol, and maleimide group of polyethylene glycol was covalently bonded to sulfhydryl group of additionally bound reduced hepatitis B antibody.

The manufacturing method of the signal amplifier is as below:

First, polystyrene bead having a diameter of 130 nm bound with carboxyl group was added by 1-Ethyl-3-[3-dimethylaminopropyl]carbodiimide hydrochloride(EDC)/Sulfo N-hydroxysulfosuccinimide(Sulfo-NHS)) to bind hydrosuccinimide group to bead, and overdose of alkaline phosphatase was made to react with bead of N-hydrosuccinimide group, whereby polystyrene bead bound by a plurality of alkaline phosphatases was manufactured. Thereafter, biotin having N-hydrosuccinimide group was covalently bonded to the polystyrene bead bound by a plurality of alkaline phosphatases.

### Second Exemplary Embodiment: Detection of Hepatitis B Antigen Using Sandwich Elisa and Verification of Signal Amplification

Hepatitis B antibody was fixed on a surface (e.g., substrate) of polystyrene, to which 100 uL (1 ng/mL) of hepatitis B antigen was added to induce antigen-antibody reaction. Thereafter, the antigen binder manufactured by the first exemplary embodiment was added by 100 uL (1 ug/mL) to react with the hepatitis B antigen, which was then washed using tris buffer solution to remove un-reacted antigen binder. At this time, 100 uL (1 ug/mL) of the signal amplifier manufactured by the first exemplary embodiment was added to allow streptavidin of antigen binder to fully bind to the biotin of signal amplifier. The un-reacted signal amplifier was washed and removed using tris buffer solution, and added by substrate solution, where absorbance at 405 nm wavelength was measured using a spectrophotometer (manufactured by Bio-rad) (Second experiment).

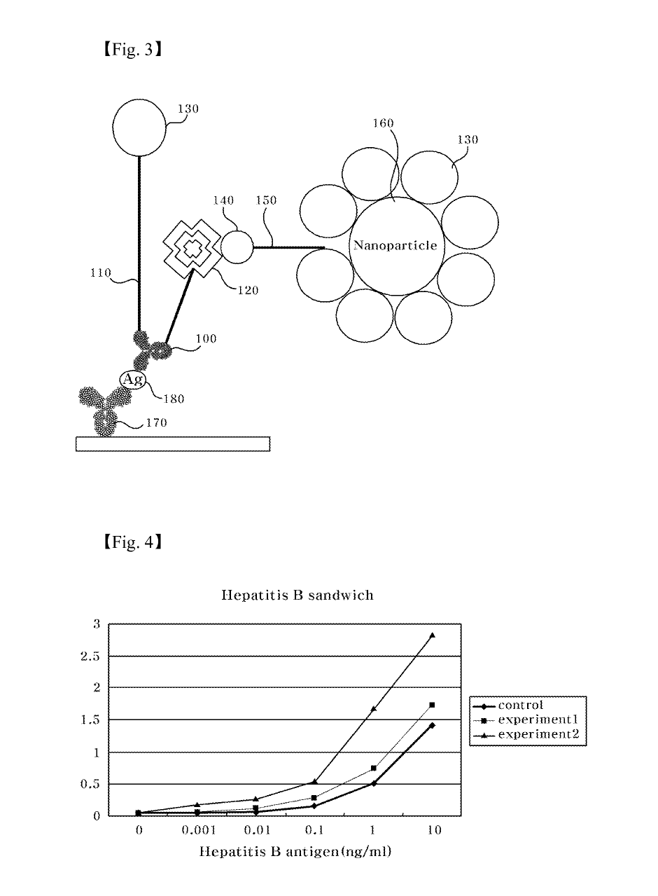

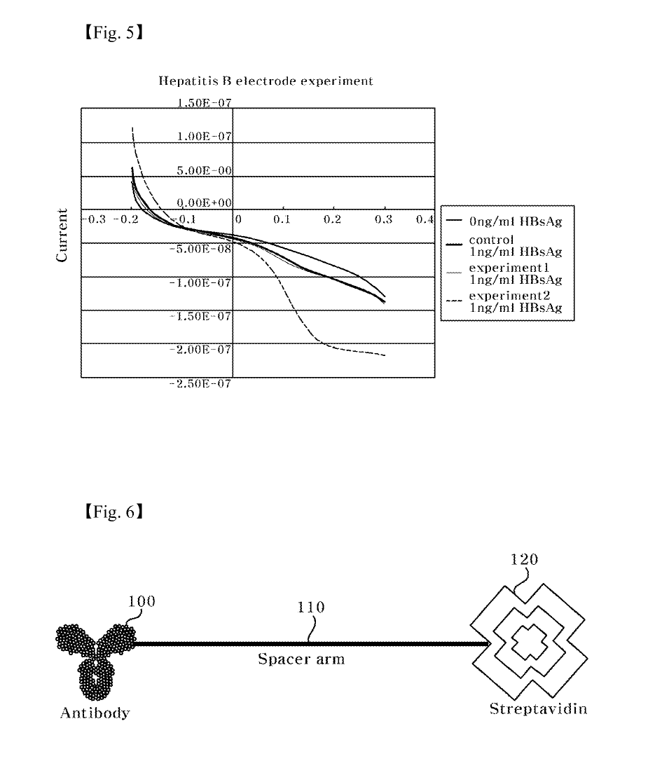

As a control group, 100 uL (1 g/mL) of hepatitis antibody bound by alkaline phosphatase was added instead of antigen binder, and as a comparative experiment, only the antigen binder was added and absorbance was measured without signal binder being added (First experiment). As a result, as shown in FIG. 4, it could be verified that the highest absorbance was recorded from an experimental group (Second experiment) where antibody binder and the signal amplifier were all reacted.

In order to electrochemically measure whether a signal was amplified, hepatitis B antibody was fixed on a surface of gold electrode, on which a micro-channel was made, and 7 uL (1 ng/mL) of hepatitis B antigen was added thereto to induce the antigen-antibody reaction. Thereafter, 7 uL (1 g/mL) of the antigen binder manufactured from the First exemplary embodiment was injected into the channel to allow reacting with the hepatitis B antibody, and un-reacted antigen binder was removed using tris buffer solution.

At this time, 7 uL (1 g/mL) of signal amplifier manufactured from the First exemplary embodiment was injected into the channel to allow streptavidin of antigen binder to fully bind to the biotin of signal amplifier. Unbound signal amplifier was washed and removed using tris buffer solution, substrate solution (10 mM of amino phenyl phosphate sodium) was injected into the channel, and an electric signal from the electrode was measured using a potentiostat (manufactured by Princeton) (Second experiment). As a control group, hepatitis antibody bound by alkaline phosphatase was added instead of antigen binder, and as a comparative experiment, only the antigen binder was added and an electric signal was measured without signal binder being added (First experiment). As a result, as shown in FIG, 5, it could be found that the highest current flow was recorded from an experimental group (Second experiment) where antibody binder and the signal amplifier were all reacted.

FIG. 6 is a mimetic diagram illustrating an antigen binder according to another exemplary embodiment of the present invention, and FIG. 7 is a schematic view illustrating a method in which a kit is used to amplify a detection signal in an immunosensor and to detect a target antigen according to another exemplary embodiment of the present invention.

Hereinafter, a method for detecting target antigen using a kit according to another exemplary embodiment of the present disclosure will be described with reference to FIGS. 6 and 7.

FIG. 6 is a mimetic diagram illustrating an antigen binder according to another exemplary embodiment of the present invention, where a first antibody (100), a first spacer (110) and streptavidin (120) are illustrated.

The first antibody (100) is an antibody binding to a target antigen, and connected to the streptavidin (120) through the first spacer (110). Furthermore, the streptavidin (120) is characterized in that it is a substance comprised of four identical molecules, and is capable of binding to biotin of one to four molecules per molecule.

The signal amplifier according to another exemplary embodiment of the present disclosure, as illustrated in FIG. 2, may include a biotin (140), a second spacer (150), a nanoparticle (160) and a detectable label (130).

The biotin (140) is a molecule configured to specifically bind to the streptavidin (120) of the antigen binder and binds to the nanoparticle (160) or the detectable label (130) through the second spacer (150). Furthermore, the nanoparticle (160) is bound by a plurality of detectable labels (130) to enable emission of much more amplified signal than that of the nanoparticle bound by a single detectable label.

FIG. 7 is a schematic view illustrating a method in which a kit is used to amplify a detection signal in an immunosensor and to detect a target antigen according to another exemplary embodiment of the present invention.

A target antibody (170) fixed to a surface (e.g., substrate) forms a complex by a target antigen (180) and antigen-antibody interaction, where if an antigen binder is brought into contact with the complex, an antigen-antibody interaction is generated by the first antibody (100) of the antigen binder and the target antigen (180).

The first spacer (110) of the antigen binder binds to the streptavidin (120), whereby a specific binding with the biotin (140) of the signal amplifier is possible. Due to the fact that the nanoparticle (160) connected to the biotin (140) through the seconds spacer (150) binds to a plurality of detectable labels (130), and the signal amplifier of several molecules (upto four molecules) is specifically binds to the antigen binder, a signal having a high sensitivity can be detected.

### Third Exemplary Embodiment: Manufacturing Method of Antigen Binder and Signal Amplifier

A manufacturing method of antigen binder is as follows:

First, one side of polyethylene glycol was bound by N-hydrosuccinimide estere group bindable with amine group, and the other side was bound by maleimide group bindable with sulfhydryl group, the streptavidin was covalently bonded to N-hydrosuccinimide group of polyethylene glycol, and maleimide group of polyethylene glycol is covalently bonded to reduced triponin-I antibody (manufactured by HiTest).

The manufacturing method of the signal amplifier is as below:

First, polystyrene bead having a diameter of 130 nm bound with carboxyl group was added by 1-Ethyl-3-[3-dimethylaminopropyl]carbodiimide hydrochloride(EDC)/Sulfo N-hydroxysulfosuccinimide (Sulfo-NHS)) to bind hydrosuccinimide group to bead, and overdose of alkaline phosphatase was made to react with bead of N-hydrosuccinimide group, whereby polystyrene bead bound by a plurality of alkaline phosphatases was manufactured. Thereafter, biotin having N-hydrosuccinimide group was covalently bonded to the polystyrene bead bound by a plurality of alkaline phosphatases.

### Fourth Exemplary Embodiment: Detection of Triponin-I Antibody Using Sandwich Elisa and Verification of Signal Amplification

Triponin-I antibody was fixed on a surface (e.g., substrate) of polystyrene, to which 100 uL (1 ng/mL) of triponin-I antigen was added to induce antigen-antibody reaction. Thereafter, the antigen binder manufactured by the third exemplary embodiment was added by 100 uL (1 g/mL) to react with the triponin-I antigen, which was then washed using tris buffer solution to remove un-reacted antigen binder. At this time, 100 uL (1 g/mL) of the signal amplifier manufactured by the third exemplary embodiment was added to allow streptavidin of antigen binder to fully bind to the biotin of signal amplifier. The un-reacted signal amplifier was washed and removed using tris buffer solution, and added by substrate solution, where absorbance at 405 nm wavelength was measured using a spectrophotometer (manufactured by Bio-rad) (Third experiment).

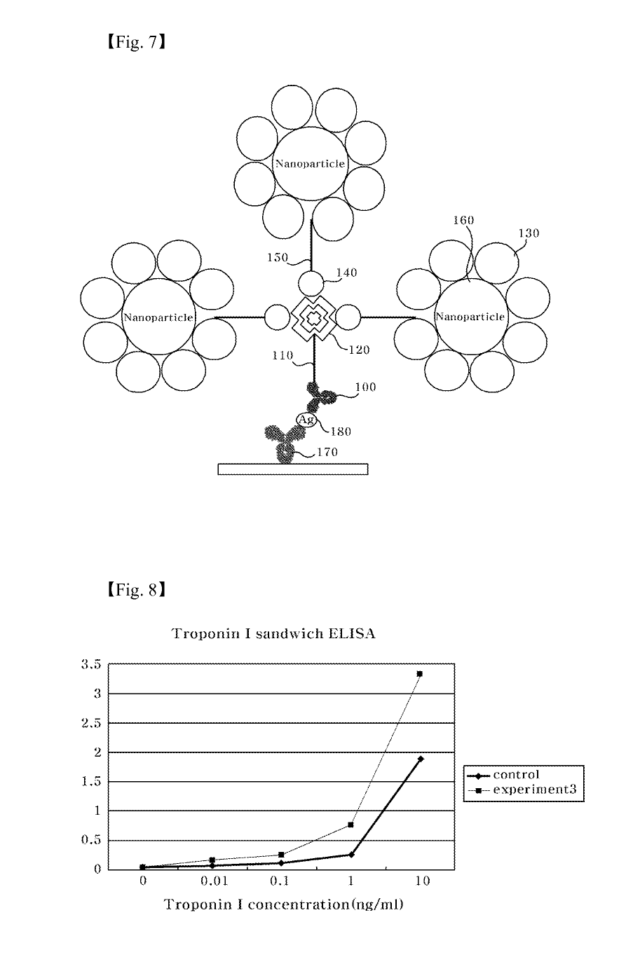

As a control group, 100 uL (1 g/mL) of triponin-I antibody bound by alkaline phosphatase was added instead of antigen binder, and absorbance was measured (Control group). As a result, as shown in FIG. 8, it could be verified that the highest absorbance was recorded from an experimental group (Third experiment) over the control group where the signal amplifier was reacted.

In order to electrochemically measure whether a signal was amplified, triponin-I antibody was fixed on a surface of gold electrode, on which a micro-channel was made, and 7 uL (1 ng/mL) of triponin-I antigen was added thereto to induce the antigen-antibody reaction. Thereafter, 7 uL (1 g/mL) of the antigen binder manufactured from the Third exemplary embodiment was injected into the channel to allow reacting with the triponin-I antigen, and un-reacted antigen binder was removed using tris buffer solution.

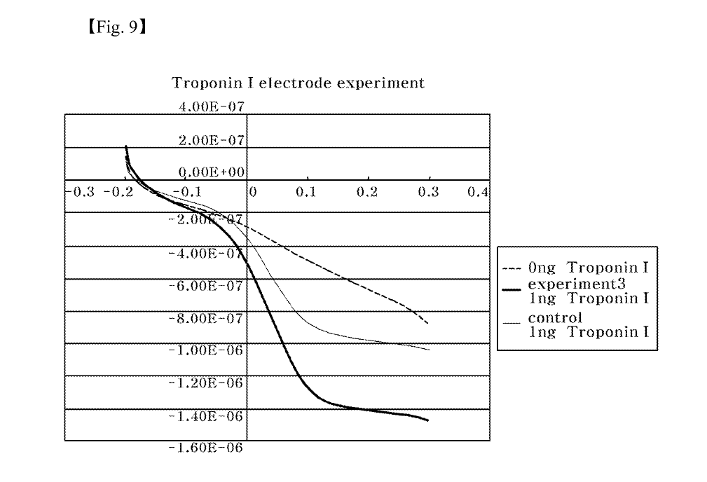

At this time, 7 uL (1 g/mL) of signal amplifier manufactured from the Third exemplary embodiment was injected into the channel to allow streptavidin of antigen binder to fully bind to the biotin of signal amplifier. Unbound signal amplifier was washed and removed using tris buffer solution, substrate solution (10 mM of amino phenyl phosphate sodium) was injected into the channel, and an electric signal from the electrode was measured using a potentiostat (manufactured by Princeton) (Third experiment). As a control group, triponin-I antibody bound by alkaline phosphatase was added instead of antigen binder. As a result, as shown in FIG. 9, it could be found that the highest current flow was recorded from an experimental group (Third experiment) over the control group where the signal amplifier was reacted.

The previous description of the present invention is provided to enable any person skilled in the art to make or use the invention. Various modifications to the invention will be readily apparent to those skilled in the art, and the generic principles defined herein may be applied to other variations without departing from the spirit or scope of the invention. Thus, the invention is not intended to limit the examples described herein, but is to be accorded the widest scope consistent with the principles and novel features disclosed herein.

As apparent from the foregoing, the kit for amplifying detected signal in immunosensor and a method for detecting target antigen using the same according to the present disclosure have an industrial applicability in that a target antigen can be effectively detected even by a small amount of target antibody to thereby reduce nonspecific detection signal and to detect an amplified signal.

1. A kit for amplifying detected signal in immunosensor, the kit comprising: an antigen binder wherein a distal end of a first spacer is connected to a first antibody, and streptavidin is connected to a portion of the first spacer or the first antibody; and a signal amplifier wherein both distal ends of a second spacer bind to biotin and nanoparticle, and the nanoparticle binds to one or more detectable labels.

2. The kit of claim 1, wherein the antigen binder is configured in such a manner that a distal end of the first spacer is connected to the first antibody, and the other distal end of the first spacer is connected to streptavidin.

3. The kit of claim 1, wherein the antigen binder is configured in such a manner that a distal end of the first spacer is connected to the first antibody, the other distal end of the first spacer is connected to a detectable label, and streptavidin is connected to a portion of the first spacer or the first antibody.

4. The kit of claim 1, wherein the streptavidin and the biotin are bound or bindable therebetween.

5. The kit of claim 1, wherein the kit includes two or more signal amplifiers for one antigen binder.

6. The kit of claim 1, wherein the kit includes four signal amplifiers for one antigen binder.

7. The kit of claim 1, wherein the streptavidin of the antigen binder binds to two or more signal amplifiers.

8. The kit of claim 1, wherein the streptavidin of the antigen binder binds to four or more signal amplifiers.

9. The kit of claim 1, wherein the nanoparticle binds to two or more detectable labels.

10. The kit of claim 1, wherein the detectable label is comprised of one or more selected from a group consisting of colored bead, antigen binder, enzyme, chromophore material, fluorescent material, phosphor material, electrically detectable molecule, molecule or quantum dot providing changed fluorescent—polarization or changed light spread.

11. The kit of claim 1, wherein the first spacer or second spacer is comprised of one or more selected from a group consisting of polyethylene glycol, polyvinyl alcohol, polyacrylic acid, polymethacrylic acid, polyacrylamide and polyvinyl pyrrolidone.

12. The kit of claim 1, wherein the first spacer or second spacer has a length of 30 Å˜60 Å.

13. The kit of claim 1, wherein the first spacer or second spacer has a length of 40 Å˜60 Å.

14. The kit of claim 1, wherein the nanoparticle has a diameter of 10 nm˜1,000 nm.

15. The kit of claim 1, wherein the nanoparticle is comprised of one or more selected from a group consisting of metal, semiconductor material and high polymer compound.

16. A method for detecting target antigen using a kit for amplifying detected signal in immunosensor, the method comprising: contacting a target antibody, a target antigen and antigen binder of claim 1; contacting a resultant thereof to a signal amplifier of claim 1; and detecting a signal generated from a detectable label of the antigen binder and the antigen binder.

17. The method of claim 16, wherein the target antibody is fixed to a plate.

18. The method of claim 16, further comprising washing the antigen binder that is not bound by the contact, subsequent to contacting the antigen binder.

19. The method of claim 16, further comprising washing the signal amplifier that is not bound by the contact, subsequent to contacting the signal amplifier.
